(19) United States
(12) Patent Application Publication
(10) Pub. No.: US 2005/0059142 A1
(43) Pub. Date: Mar. 17, 2005

(54) Apparatus for aerobic liquid-phase fermentation

(76) Inventors: Alexander Vinarov, Moscow (RU); Dmitriy Sokolov, Moscow (RU); Vladimir Smirnov, Moscow (RU); Elena Sokolova, Moscow (RU); Dmitriy A. Vinarov, Fitchburg, WI (US)

Correspondence Address: Dmitriy A. Vinarov, 3015 Rothmore Lane, Fitchburg, WI, 53711, US

(21) Appl. No.: 10/892,660

(22) Filed: Jul. 16, 2004

(30) Foreign Application Priority Data
Jul. 24, 2003 (RU) 2003122732

(51) Int. Cl. C12P 7/06; C12M 1/02

(52) U.S. Cl. 435/295.1

(57) ABSTRACT

The present invention describes the apparatus for aerobic liquid-phase fermentation. The present invention relates to the field of chemical, physical and physical-chemical processes carried out in apparatus with an aeration and intermixing of a liquid medium, namely: processes of biosynthesis of various biological products, processes of processing waste from various productions, processing of semi-products, and processes of clearing of waste water that could also can be used in food, medical, microbiological, petrochemical industries, and also in an ecological remediation of an environment from various wastes.

### CROSS-REFERENCE TO RELATED APPLICATIONS

RU #2003122732. Priority Date: Jul. 24, 2003

### STATEMENT REGARDING FEDERALLY SPONSORED RESEARCH OR DEVELOPMENT

Not Applicable

### TECHNICAL FIELD

The present invention relates to the field of chemical, physical and physical-chemical processes carried out in an apparatus with aeration and intermixing of a liquid medium, namely: processes of biosynthesis of various biological products, processes of processing waste from various productions, processing of semi-products, and processes of clearing of waste water that could also can be used in food, medical, microbiological, petrochemical industries, and also in an ecological remediation of an environment from various wastes.

### BACKGROUND OF THE INVENTION

The different constructions of apparatuses—fermenters, bioreactors for realization of aerobic fermentation processes on various mediums with application of various microorganisms are known. The U.S. Pat. No. 3,957,585 describes fermenter with centrally located diffuser and intermixing device, wherein an aerial-liquid turbine is inside a diffuser. The apparatus described in that patent is supplied with the tubular heat exchanger located in a top of a diffuser and the mechanical foam-breaker device for foam-destruction. For additional vertical circulation and best intermixing of aerated medium the apparatus is supplied with the circulating pump ensuring an external outline of circulation. The apparatus is intended for liquid-phase aerobic processes of fermentation and ensures a required level of oxygen mass-transfer and mixing of a medium. However, the disadvantage of this apparatus is that it is necessary to refer the limited enough zone of turbulence of a medium that does not allow application of given construction to large volumes (more than 100-200 m³) and also high specific consumption of energy. So, on 1 kg transferred in a fermentation medium of oxygen in such apparatus is expended not less than 0.6-0.9 kWh of the electric power.

With the purpose of acceleration of mass-transfer processes in the U.S. Pat. No. 4,752,564 an apparatus for production of microbial cells, representing a closed vessel with feeders of a mineral feed, source of oxygen, source of carbon, monitoring and foam-regulation, and also heat-exchanger device was proposed. By design the intensification of the fermentation process is ensured at the expense of additional introduction in a construction of a apparatus of the vibration device, influencing on cells with certain frequency and intensity. As disadvantage of a known apparatus is that it is only efficient for small volumes and sterile fermentation processes.

In the U.S. Pat. No. 4,204,042 the method for aeration and intermixing realized in an apparatus consisting from two informed tanks is presented. By design sterile air discretely injected in the form of bubbles into tanks that ensures pulsate effect and acceleration process of microorganisms growth. As limitation of an offered apparatus it is necessary to recognize it efficiency only for well soluble substrates and impossibility of maintenance on scale up to large volumes sufficient turbulence effect of a fermentation medium.

Other principle of aeration and intermixing of a fermentation medium is realized in the GB patent #1 353008. The apparatus, described in that patent, is made from two vertically located vessels, between which the circulation by whole fermentation medium is organized on the basis of a difference of denseness of aerated and de-aerated liquid. By design aeration realized by using babbler, located in the bottom of one vessels, and the exhaust gas, separated from a liquid after degassing process, leaves through the upper tube on other vertical vessel, on which downwards circulates degassed liquid, passing the heat-exchanger device. The apparatus is intended for work with large volumes of a fermentation medium. The disadvantage of an apparatus is in its insufficient turbulence of a fermentation medium, that does not allow effective use of poor-soluble substrates and dispersible mediums for fermentation processes, and also rather low surface of contact of phases a gas—liquid, stipulated by coalescence of raising bubbles. In this connection, the velocity of mass-transfer in such apparatus does not exceed 3-4 kg O₂/m³ per hour that it is not enough, for example, for high-productivity processes of deriving of a microbe biomass.

In the U.S. Pat. No. 4,656,138 describes a fermenter, which has aerator and mixing device, circulating tube and diaphragm, and also camera for distribution of a gas and liquid streams. The cavitation waves, generated in an apparatus promote of intensification of process. However, the offered fermenter is complicated for scaling, and, besides it is poorly effective for large volumes, for example, 100 and more m³. Furthermore, the problems of foam breading have not been solved.

In the economic patent DD #59549, the construction of column fermenter of the jet type, having located on height sections, connected by overflowing tubes in which are bridged gaseous branch pipes ensuring by the mean of an injection supply of a fresh air on an aeration is presented. Vigorous mixing of a medium in this apparatus realize by an external circulating outline with the pump. The fermenter can be realized on large volumes 300 m³ and more.

The disadvantage of a known fermenter is its significant energy consumption, connected with pumping of liquid mass in a vertical vessel by high-pressure pump. By design the specific energy consumption on 1 kg O₂ in this fermentor makes not less than 0.7 kWh. Furthermore the problem of the foam-generation, characteristic for many fermentation mediums has not been solved.

The apparatus for aerobic liquid fermentation on soluble and dispersed mediums with use of microorganisms, utilizing carbon containing substrate and providing mixing and aeration of a fermentation medium was considered by Olsen A. J. “Manufacture of bakers yeast by continuous culture”, Chem. Ing. 1960, 416p. The fermenter contains devices for maintaining temperature of a medium, pH of a medium, concentration of the dissolved oxygen, level of foam, level of a liquid, and also devices for supplying of some necessary components of a carbon feed for microorganisms living and growth, in particular, melasse. In this apparatus the devices for supplying of aerated gas by apparatus of its inlet in the lower air camera and dispersions through air tubes, and also the device for outlet of a fermentation medium and branch pipes for a mineral components supplying and feeding of necessary for process components, in p articular, acid, detergents, etc. are provided. In a construction of this apparatus the heat exchanger made as the jacket at the bottom of cylindrical vessel also is maked in addition. The construction of the apparatus can be used for large volumes, however with magnification of sizes the efficiency of oxygen mass-transfer and effect of turbulence of a medium for want of intermixing is reduced, the average size of air bubbles is increased. In this connection, efficiency and the productivity of processes of aerobic fermentation in such apparatus with magnification of its volume are reduced.

The achievable parameters in such apparatus make (for volume 100 m³): a velocity of oxygen mass-transfer 2.5-3.5 kg O₂/m³ hour, and specific energy consumption of 0.6-0.7 kWh/kg O₂, that determines it low technological efficiency.

### BRIEF DESCRIPTION OF THE DRAWINGS

FIG. 1 shows the general view (sectional view) of an apparatus for aerobic liquid-phase fermentation.

FIG. 2 shows the top view of variant of an apparatus for aerobic liquid-phase fermentation with three axial pumps.

FIG. 3 shows the top view of variant of an apparatus for aerobic liquid-phase fermentation with six pumps.

FIG. 4 shows the vertical sectional view of the realization of the proposed apparatus with the geometric volume of 50 m³.

FIG. 5 shows the vertical sectional view of the realization of the proposed apparatus with the geometric volume of 300 m³.

### DETAILED DESCRIPTION OF THE INVENTION

The technical problem solved by an offered apparatus for aerobic liquid fermentation, consists in increase of its efficiency at the expense of maintenance of a high degree of a dispersion of aerated gas in a liquid, effective turbulence of a medium and increase of a velocity of oxygen mass-transfer to decrease specific energy consumption on the oxygen transport in a fermentation medium.

The technical result received for realization of the given apparatus, consists in a reduced energy consumption on process of aerobic fermentation, increase of productivity and yield of microbiological process, and also possibility of creation of industrial apparatus of proposed construction of various volume, including up to 100-300 m³ and more.

For reaching indicated technical result it is proposed to use an apparatus for aerobic liquid fermentation on soluble and dispersible mediums with use of microorganisms, utilizing various carbon substrates, and providing intermixing and an aeration of a fermentation medium, and having devices for maintaining temperature of a medium, pH of a medium, concentration of the dissolved oxygen, foam level, level of a liquid, and also devices for feeding of some components with a carbon and mineral nutrition, necessary for habitability of microorganisms, for aerated gas, for removing of a fermentation medium and for foam-breaking, and representing a vessel with a cover and bottom, supplied with branch pipes for inlet and outlet of necessary components for process of aerobic liquid fermentation and the heat exchanger, and in a central part of vessel body, having the cylindrical form making a recess at which walls are tangentially located of one or more tubes, wherein the inlets are connected with volume of vessel, and the outlets are connected with additionally entered devises for pumping over of a liquid, and the indicated outlets devises for pumping over of a liquid are connected by branch pipes to inlets of ejectors, each of ejector has in its inlet section more than one coneffusers and is supplied with a branch pipe for inlet feeding of aerated gas, and the outlets of ejectors constructed as a diffuser, are located at the bottom of a surface of vessel and are directed tangentially concerning a vertical axes of vessel body. Preferably, ejectors have some coneffusers with an adjacent angle from 15 up to 40 degrees, and the part of inlet free section for passing of a liquid flow makes from 20% up to 70%, and it is most preferable from 40% up to 60%. Usually, ejectors are connected by branch pipes to outlets of devises for pumping over of a liquid in parallel and each ejector has a branch pipe for inlet of aerated gas. However, the variant is possible wherein ejectors are connected by branch pipes to outlets of devices for pumping over of a liquid sequentially and each ejector has a branch pipe for inlet of aerated gas. Mainly, ratio of a diameter of inlet tube for passing of a liquid flow in ejector and diameter of inlet tube of coneffusers consists of 0.01-0.1, but it is most preferable, a ratio of a diameter of inlet tube for passing of a liquid flow in ejector and diameter of inlet tube of coneffusers 0.02-0.2. Usually branch pipes for inlet of aerated gas in ejector are connected to atmosphere. But it is possible that branch pipes for inlet of aerated gas in ejector are connected to outlets of means for sterilization of aerated gas, which inlets are connected to outlet devices for dispensing of aerated gas. Preferably outlet of each ejector is directed in direction of tubes located at walls of recess of vessel body. In preferable variant of realization indicated devices for pumping over of a liquid are constructed as axial pumps of low pressure. Usually in a apparatus the heat exchanger, made as plates or pipes, located with a clearance on an internal cylindrical surface of an apparatus and/or on a bottom, except for a central part of the vessel with recess is used. In the most preferable variant the heat exchanger is located outside of a body of the vessel and is connected with apparatus by a circulating outline. Main cover of an apparatus is made conic and is supplied with a controlled means for removal of exhaust gas. Preferably in this case realizations of an outlet of controlled means for removal of spent gas are connected to inlet of the means for clearing of exhaust gas. In the most preferable variant of realization of an apparatus the device for foam-breading is made as a gauze located horizontally with respect to vertical axes of the vessel, and at a distance from the bottom consists from 0.5-0.67 height of a cylindrical part of body of an apparatus. In this case indicated device for foam-breading can in addition contains one or more branch pipes for intake of foam, located above gauze and connected to one or several branch pipes for inlet of aerated gas in ejector. Structurally an apparatus is made with possibility of using in process of aerobic fermentation of yeast, fungi, and bacteria. In any form of realization the proposed apparatus is intended for fermentation processing of liquid mediums containing spirits, sugar, hydrocarbon and organic acids.

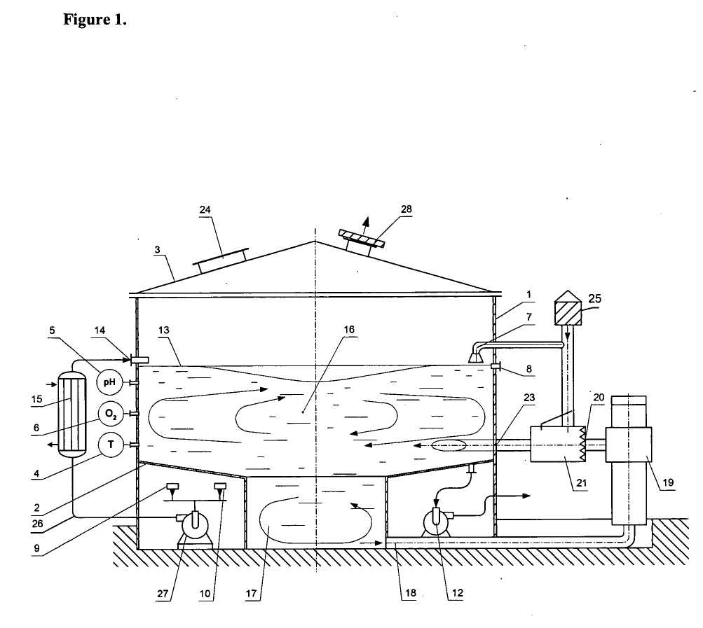

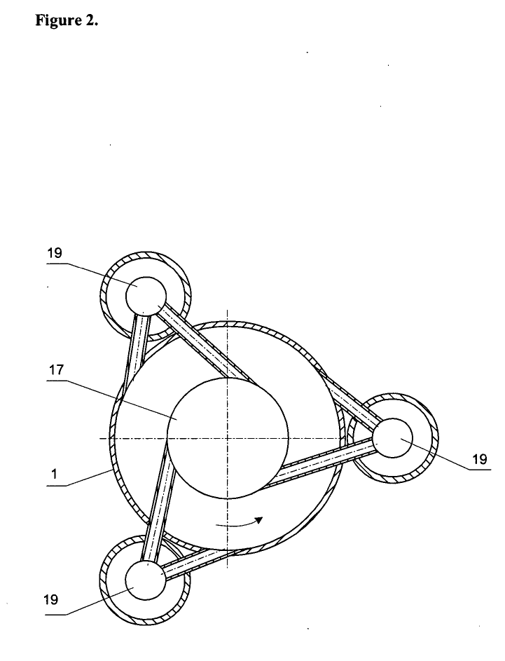

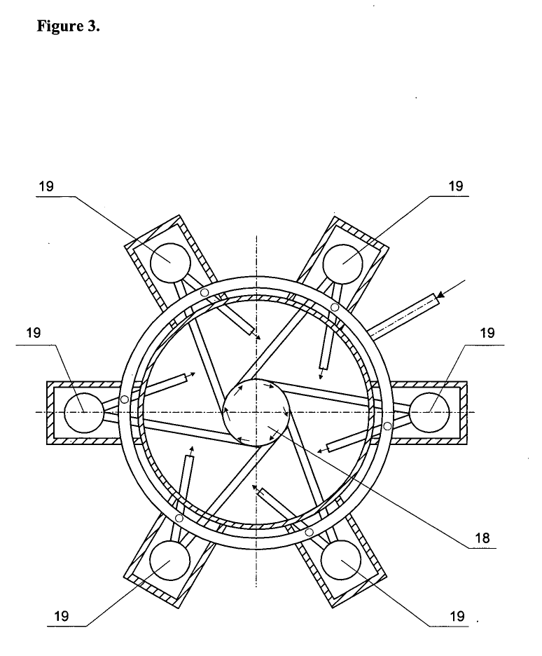

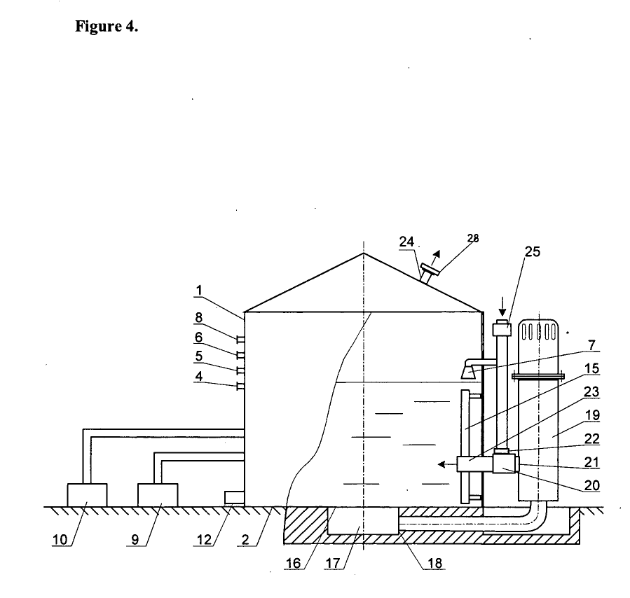

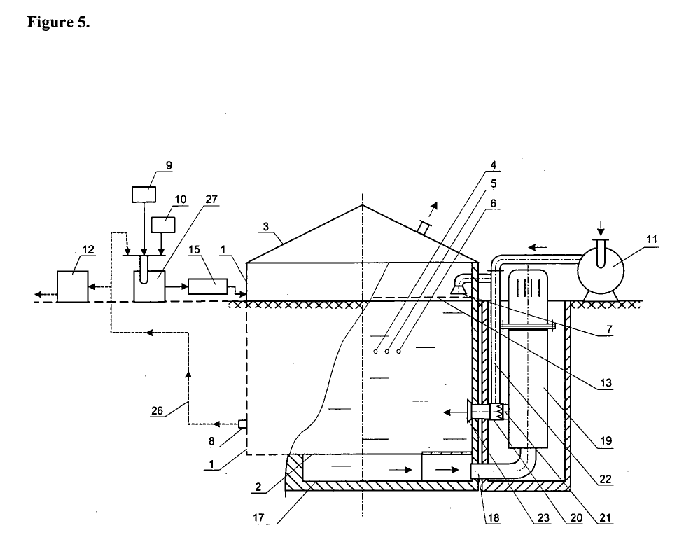

Therein after proposed apparatus for aerobic liquid-phase fermentation on soluble and dispersible mediums will be considered with the references to an illustrative material, where: on FIG. 1 the general view (sectional view) of a apparatus is indicated; on FIG. 2 the top view of variant with three axial pumps is indicated; on FIG. 3—top view of variant with six pumps is indicated; on FIG. 4 and FIG. 5—the vertical sectional view of a typical apparatus in various variants of realization are indicated. On a graphic material the following legends are used: body 1, bottom 2, cover 3, device 4 for maintaining of temperature of a medium, device 5 for maintaining pH of a medium, device 6 maintaining of concentration of the dissolved oxygen, device 7 for maintaining of a level of form, device 8 for maintaining of a level of a liquid, device 9 for feeding of necessary components of carbon nutrition for microorganism growth, device 10 for feeding of necessary components of mineral nutrition for microorganism growth, device 11 for supplying of aerated gas, device 12 for outlet of fermentation medium, device 13 for foam-breading, branch pipes 14 for inlet and outlet of necessary components from process of liquid-phase aerobic fermentation, heat exchanger 15, central part 16 of apparatus body, recess 17 at walls of a central part, pipe 18, pumps 19 for liquid, ejector device 20, coneffuser 21, branch pipe 22 for inlet of aerated gas in ejector device 20, diffuser 23, valve 24, air-sterilizing filter 25, circulating outline 26, pump 27, biofilter 28.

In one realization of the proposed apparatus the geometric volume 50 m³ is made as follows (FIG. 4): the cylindrical body 1 with a bottom 2 and cover 3 is completely made from a stainless steel and has in the lower central part 16 recesses 17, at which wall tangential to one pipe 18 also made from a stainless steel is located, wherein an outlet is connected with pump for liquid 19, the axial pump, have productivity 2500 m³/hour of a transferable liquid and a hydrostatic head −3.0 m of water column. The outlet of pump 19 is connected by metal pipes with a diameter of 250 mm to an inlet in ejector device 20, which is located in parallel and having in a inlet section coneffusers 21 with an entrance diameter of 5 mm and adjacent angle 20 degrees with a free section for pass of a liquid of 45%. In each ejector device 20 the Teflon branch pipe 22 for inletting of aerated gas—air, connected with outlet from an air-sterilizing filter 25 is constructed, that ensures inlet of a sterile air for aeration in an apparatus through ejector device 20 as small dispersed bubbles. The outlets from two ejector devices 20 are made as diffusers 23 and are located at the bottom of the body 1 of apparatus tangentially and opposite against a direction of pipes located at walls of recess 17 of body 1 of apparatus, that ensures effective turbulence and dispersion of phases. The apparatus is supplied with the plate-like heat exchanger 15, located at an internal cylindrical surface of apparatus body 1. The apparatus is also supplied with the valve 24 for adjustable removal of exhaust gas, which is connected to biofilter 28 for clearing of outlet gas.

The apparatus contains the device 5 for maintaining pH of a medium, device 4 for maintaining of temperature of a medium, device 6 for maintaining the dissolved oxygen concentration, device 7 for maintaining a level foam, device 8 for maintaining liquid level, and also devices 9 and 10, which are dosing pumps for carbon and mineral components supplied, and device 12 for outlet of fermentation medium from a apparatus. The apparatus is intended for realization periodic or continuous liquid-phase fermentation on mediums containing sugar, spirits, hydrocarbon and organic acids with application of yeast, bacteria or fungi.

The apparatus with geometric volume 300 _{M}³ is made (FIG. 3 and FIG. 5) as a metal-concrete construction by a diameter 8.0M with recess 17 in a bottom 2 in the lower central part of body 1 with diameter 3.0 M and depth 1.0 M, which is located below the ground level, and in it there are six polychlorovinyl pipes 18 tangentially located, which supply inlet of liquid from volume of a apparatus to pumps 19 which are six axial pumps with productivity 3000 M³/hour of liquid each and pressure 5 M of water column. The outlet of each pump 19 is connected by a silicone branch pipe with a diameter of 500 mm to one ejector device 20, having in the inlet section coneffusers 21 with an entrance diameter of 20 mm and adjacent angle 35 degrees, and the part of inlet free section for pass of a liquid makes 55%. In four out of six ejector devices 20 polychlorovinyl pipes 22 are constructed connected with the device 11 for supply of air for aeration. The outlet for each out of six ejector devices 20 is made as a diffuser and it is located tangentially in the lower cylindrical part of the apparatus body 1 and in opposite direction to pipes 18 for outlet of a liquid from recess of the apparatus. The apparatus is supplied with the tubular heat exchanger 15, located outside of tank body 1 and connected with an apparatus by a circulating outline 26 with the centrifugal pump 27 for circulation of a medium through the heat exchanger 15 into body 1 of apparatus. At height 0.67 from height of a cylindrical part of apparatus body 1 the device 13 for foam-breading as a metal gauze is located, and above it two branch pipes from a stainless steel for tapping of unduly forming foam are constructed, and their outlets are connected to inlets of branch pipes 22 for inlet of aerated gas, which are constructed in remaining two ejector devices 20. The apparatus also contains device 4 for maintaining of temperature of a medium, device 5 for maintaining pH of a medium, device 6 for maintaining of concentration of the dissolved oxygen, device 7 for maintaining of a foam level, device 8 for maintaining of liquid level, device 9 for feeding of necessary components of carbon nutrition for microorganism growth, device 10 for feeding of necessary components of mineral nutrition for microorganism growth, device 12 for outlet of fermentation medium. The apparatus is intended for aerobic clearing processes of wastewater from various manufactures, for fermentation processing of alcohol fabric waste, for fermentation processing of wood hydrolyzates and vegetative raw material.

The proposed construction of an apparatus in any one of above discussed variations works as follows. At first mineral and carbon components of a nutritious medium in a water solution are feed into apparatus body 1 through branch pipes 14 and by using devices 9 and 10. With the use of the device 4 for maintaining of temperature of a medium, device 5 for maintaining pH of a medium, device 6 for maintaining a concentration of the dissolved oxygen, conditions, which are set prior to inputting of initial microorganisms used for desired processing of raw material are created, and volume of recess 17 central parts of a apparatus with use of the circulating pump 27, connected through a circulating outline to a apparatus is filled. Initial culture of microorganisms from vessel for cultivation and/or from storage for pure culture of microorganisms by using the pump 27 is fed in apparatus. Pumps for liquid 19, on which inlets by use branch pipes 18 fermentation medium from the recess 17 of apparatuses arrives, are turned on and an aeration and intermixing of the indicated medium with use ejector devices 20 having coneffusers 21 are realized.

The air for the aeration arrives into ejector device 20 on branch pipes 22 through sterilizing filters 25 and as a small finely dispersible gas-liquid emulsion through outlet diffusers 23, located at internal walls of body 1 of apparatus tangentially cames out. By use of kinetic energy of gas-liquid jets rotation, aeration and turbulence of a nutritious medium in an apparatus is ensured. In accordance with increase of concentration of microorganisms in volume of recess 17 of apparatus solutions of nutrient components by the pump 27 are feed into a apparatus and volume of a liquid in a apparatus is raised through a level, which given by device 8 for level, with using pumping over of a medium by circulating outline 26 through the heat-exchanger 15, maintaining of temperatures of a medium in a apparatus which given by the device 4 is ensured. The device 13 for foam-breading is located above a level of a liquid and made as gauze ensures stabilization of foam-generation with aid of rotary movement of a liquid at a surface of gauze. In case of abundant foam-generation of fermentation medium the excessive foam is deleted with use of devices 7, made as intaking branch pipes and connected to a branch pipe 22 of an inlet air in ejector device 20. The spent air leaves from an apparatus through a biofilter 28 into the atmosphere.

In a continuous fermentation process in a apparatus water solutions of components of mineral and carbon feed through devices 9, 10 and circulating pump 27 are constantly entered, and outlet of fermentation medium with use of the device 12, which is preferably used for dosing the pump are also constantly realized.

For desired use of an apparatus for cleaning of wastewater the polluted stream continuously arrives through the circulating pump 27 in body 1 of apparatus, where process of utilization of contamination is carried out through habitability of aerobic population of microorganisms. The cleared stream is continuously removed from an apparatus.

Furthermore, proposed construction of an apparatus will be considered with use of concrete examples of realization of microbiological processes, which can be carried out on anyone of the variants of realization of an offered apparatus.

### EXAMPLES

### Example 1

In a apparatus of the indicated construction with geometric volume 50 m³ a periodic process of microbiological synthesis of a citric acid is realized. As a microbial culture producing the citric acid the strain of fungi Aspergillus Niger is used. Culture is grown on a nutritious medium containing: a chloride ammonium−2 g/L, phosphate of potassium monobydrate−1.5 g/L, sulfuric zinc×7 H₂O−0.005 g/L, sulfuric magnesium×7 H₂O−0.5 g/L, and also as a source of carbon—raw sugar which is inputted during of periodic process discretely in total quantity of 10% by vol. Intermixing and aeration of a medium in a apparatus are realized by supplying of gas-liquid stream from ejector device in volume of a apparatus, therewith the circulation period of a medium through ejector device is two minute. A level of an aeration of a medium during of air supplying, passing an air filter, through ejector device consists 1.1 m³/m³/min. The medium temperature of 30° C. is sustained in an apparatus by circulation of a medium through the external tubular heat exchanger. Effective intermixing of all volume of a medium and dispersion of bubbles of an air ensures intensive accumulation of a citric acid and in four day after entering of initial culture in an apparatus the concentration of a citric acid achieved is 100-105 g/L.

### Example 2

In a apparatus of indicated construction with volume of 100 m³ a continuous biosynthesis process for protein-vitamin product production by using bacterial cultures Propionibacterium freundenreicheii and Lactobacillus cassei and carbohydrate-containing substrate of the form of concentrate whey containing 14-16% of dry substances is carried out. The cultivation is carried out at the temperature of 32° C. and pH=6.6 for desired specific rate of a medium flow 0.09 h⁻¹. Aeration and intermixing of a medium are realized with three ejector devices connected to three axial pumps with productivity of each pump of 2000 m³/h. The output biosuspension from fermentation process is carried away from an apparatus continuously and fed to the next stages, including drying process. In a final product the contents of a protein makes 25% of dry substance.

### Example 3

In an apparatus of the indicated construction with geometric volume 160 m³ a continuous process of fermentation and deriving of protein fodder product is carried out by using starch-containing substrate—vegetative raw material, namely, flour of rye, previously passed of heat-reagent processing. The yeast strain Saccharomycopsis fibuligera is applied as the strain for protein production. The nutritious medium contains: flour—10% by weight, ammonium sulfate 10 g/L, potassium phosphate 2.0 g/L, magnesium sulfate 1.0 g/L. The cultivation process is carried out with the specific rate of a medium 0.1 h⁻¹, temperature 33° C. and pH=5.5. Turbulence and aeration of a medium are ensured by using inlet of gas-liquid flow from four ejector devices, connected to four axial pumps, with productivity of each pump of 2500 m³/h. The level of an aeration of a medium is 1.5 m³/min. The polymeric gauze for prevention redundant foam generation in a above a level of a liquid with volume 100 m³ apparatus is constructed and above which the branch pipe for putting out of excessive foam to an inlet of a branch pipe for aerated air from ejectors is located. The productivity of an apparatus is 20 t/day (in recalculation on a dry product) of a fodder product containing 45% of a protein, vitamins of group B and microelements. The yield of a product from initial raw material is 82%. Specific energy consumption in fermentation process is 0.35 kWh/kg of produced protein product.

### Example 4

In an apparatus of the indicated construction with volume of 300 m³ a continuous process of microbiological processing of alcohol production waste is carried out with deriving fodder protein product. Initial distiller's soluble represents a dispersible medium and contains: fermentated carbohydrates (dextrins)—1.5%, not fermentated carbohydrates (pentosans)—2.0%, fiber—3%, organic acids—0.5%, ethanol spirit—1.2%, all of dry substances in distiller's soluble—5.9%. In fermentation medium an ammonium sulfate in concentration 5 g/L, ferrous sulfate seven-water 0.1 g/L, zinc sulfate seven-water 0.05 g/L are added. The association of cultures contains fungi Endomycopsis fibuligera, yeast Pichia carsonii and bacteria Rhodococcus erythropolis for protein production is used. The fermentation process is carried out continuously with the specific rate of a medium 0.08 h⁻¹, supporting temperature 32-34° C. and pH=5.0. The body of an apparatus is made from stainless steel, and the lower recess is located below than level of ground. The apparatus is supplied with four ejector devices connected to four axial pumps, and also internal heat exchanger located in the lower cylindrical part of a tank. The productivity of an apparatus (based on a dry product) is 25 t/day, and content of protein in a product is 55%. Specific energy consumption in fermentation process consists of 0.33 kWh per kg of product.

### Example 5

In an apparatus of the indicated construction with geometric volume of 400 m³, made from ferroconcretes and supplied with six ejector devices and six axial pumps process of aerobic biological cleaning of waste water is carried out. The wastewater of an industrial production contains phenol, ethyl acetates, methanol, acetic and hydrocarbon contaminants. A natural association of microorganisms is used during the cleaning process. The value of COD (Chemical Oxygen Demand) in waste water, arriving on clearing, makes 3000 mgO₂/L. Process is carried out continuously at the temperature of 28-30° C. and with specific rate of medium flow 0.03 h⁻¹. Achievable in one apparatus in a continuous process the degree of cleaning calculated on the bases of reduction of the value of COD is 98%.

The presented examples do not limit possibilities of application of an offered apparatus for a realization of processes of liquid fermentation.

In an apparatus of proposed construction the high velocity of mass-transfer of oxygen is ensured due to intensive turbulence of a medium and dispersion of air and also the high degree of cleaning of contamination from waste water or the high yield of target product are achieved.

All publications cited herein are hereby incorporated by reference in their entirety. In the case of conflict between the present disclosure and the incorporated publications, the present disclosure should control.

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

1. The apparatus for aerobic liquid-phase fermentation on soluble and dispersible mediums with use of microorganisms, capable of utilizing various carbon-containing substrates providing intermixing and an aeration of a fermentation medium, and having devices for maintaining temperature of a medium, pH of a medium, concentration of the dissolved oxygen, level of foam generation, level of a liquid, and also feeding devices necessary for growth of microorganisms components of a carbon and mineral nutrition, for aerated gas, for removing of a fermentation medium and for foam-breaking, and representing a vessel with a cover and a bottom, supplied with branch pipes for inlet and outlet of necessary components for aerobic liquid-phase fermentation process and the heat exchanger is characterized in that in a central part of vessel body, having the cylindrical form is made a recess, at which walls one or more tubes are located tangentially, which inlets are connected with volume of vessel, and the outlets are connected with additional devices for pumping over a liquid, wherein the outlets indicated devices for pumping a liquid are connected by branch pipes to inlets of ejector devices, and each of ejector device has in its inlet section more than one coneffusers or cone-shaped tubes, and it is supplied with a branch pipe for feeding of aerated gas, and the outlets of ejector devices constructed as a diffuser, are located at the bottom of a surface of vessel body and are directed in tangential direction concerning a vertical axes of vessel body.

2. The apparatus according to claim 1, wherein the ejector devices have coneffusers with an adjacent angle from 15 up to 40 degrees, wherein the inlet free section for passage of a liquid flow consists from 20% up to 70%.

3. The apparatus according to claim 2, wherein the part of inlet free section for passage of a liquid flow consists preferable from 40% up to 60%.

4. The apparatus according to claim 1, wherein the ejector devices are connected by branch pipes to outlets of devices for pumping over of a liquid in parallel and each ejector device has a branch pipe for inlet of aerated gas.

5. The apparatus according to claim 1, wherein the ejector devices are connected by branch pipes to outlets of devices for pumping over of a liquid sequentially and each ejector device has a branch pipe for inlet of aerated gas.

6. The apparatus according to any of claims 1 to 5, wherein the ratio of a diameter of inlet tube for passing of a liquid flow in ejector device and diameter of inlet tube of coneffusers is 0.01-0:1.

7. The apparatus according to claim 6, wherein the ratio of a diameter of inlet tube for passing of a liquid flow in ejector and diameter of inlet tube of coneffusers is 0.02-0.2.

8. The apparatus according to any of claims 1 to 7, wherein the branch pipes for inlet of aerated gas in ejector device are connected to atmosphere.

9. The apparatus according to any of claims 1 to 8, wherein the branch pipes for inlet of aerated gas in ejector are connected to outlets as means for sterilization of aerated gas, which inlets are connected to outlets of devices for dispensing of aerated gas.

10. The apparatus according to any of claims 1 to 9, wherein the outlet of each ejector device is directed in contrary direction with respect to tubes located at walls of recess of vessel body.

11. The apparatus according to any of claims 1 to 10, wherein the indicated devices for pumping over of a liquid are constructed as axial pumps of low pressure.

12. The apparatus according to any of claims 1 to 11, wherein the heat exchanger, made as plates or pipes, located with a clearance on an internal cylindrical surface of an apparatus and/or on a bottom, except for a central part of the vessel body with recess.

13. The apparatus according to claim 12, wherein the heat exchanger is located outside of body of the vessel and is connected with apparatus by a circulating outline.

14. The apparatus according to any of claims 1 to 13, wherein the cover is made conic and is supplied with a controlled means for removal of exhaust gas.

15. The apparatus according to claim 14, wherein the outlet of a controlled means for removal of spent gas is connected to an inlet of the device for cleaning of exhaust gas.

16. The apparatus according to any of claims 1 to 15, wherein the device for foam-breading is made as a gauze located horizontally with respect to vertical axes of the vessel body, and its distance from the bottom consists from 0.5 up to 0.66 heights of a cylindrical part of body of an apparatus.

17. The apparatus according to claim 16, wherein the device for foam-breading in addition contains one or more branch pipes for intake of foam, located above a gauze and connected to one or several branch pipes for inlet of aerated gas in ejector devices.

18. The apparatus according to any of claims 1 to 17, wherein it is made with possibility of using in process of aerobic fermentation with yeast, fungi, bacteria.

19. The apparatus according to any of claims 1 to 18, wherein it is intended for fermentation processing of liquid mediums containing spirits, sugar, hydrocarbon, organic acids.
